(19) United States
(12) Patent Application Publication
(10) Pub. No.: US 2017/0020396 A1
(43) Pub. Date: Jan. 26, 2017

(54) Gastric Tube Temperature Sensing Apparatus

(71) Applicants: James Boone, III, El Paso, TX (US); Jean Jean-Pierre, El Paso, TX (US)

(72) Inventors: James Boone, III, El Paso, TX (US); Jean Jean-Pierre, El Paso, TX (US)

(21) Appl. No.: 14/806,347

(22) Filed: Jul. 22, 2015

(51) Int. Cl. A61B 5/01; A61J 15/00; A61B 5/00

(52) U.S. Cl. CPC A61B 5/01; A61B 5/687; A61J 15/0084; A61J 15/0092; A61J 15/0096

(57) ABSTRACT

A gastric tube temperature sensing apparatus for use during gastric or surgical procedures on a patient includes a gastric tube, such as an NG or OG tube, having a proximal end opposite a distal end and defining a tubular configuration. The gastric tube includes a flexible construction and defines a plurality of spaced apart openings proximate the distal end. An esophageal temperature device includes a temperature sensor situated in the gastric tube proximate the distal end thereof and a temperature monitoring element operatively connected to and displaced from the temperature sensor. The temperature monitoring element is situated external from the gastric tube and in communication with the temperature sensor, such as by wires. The temperature monitoring element may be electrically connected to a vitals monitor or the like. The apparatus includes an adapter for coupling a proximal end of the gastric tube to another device such as a suction pump.

### BACKGROUND OF THE INVENTION

This invention relates particularly to a gastric tube having dual function, an integrated temperature sensing mechanism that can digitally record temperature on a monitoring device and a gastric suctioning apparatus for aspiration of stomach contents. The gastric suctioning portion of the apparatus includes a tethered adaptor so as to eliminate the tendency of being misplaced or lost. Further, the gastric tube may be used for feeding when indicated.

The overall intent of this invention is to have a multifaceted apparatus that would be very useful in patients with prolonged hospital stay such as intensive care units, chronic ventilator dependency, nursing homes, rehabilitation units and patients undergoing surgical procedures. Since this gastric tube is typically threaded trough the oral or nasal cavity, it is very undesirable to crowd such limited spaces with additional tubes. Refraining from doing so may minimize the incidence of injury or ulceration in the oral and nasal cavity.

Currently, the aforementioned categories of patients, surgical patients under general anesthesia, or any others who need gastric suctioning (in order to reduce the risk of aspiration of gastric content into the lungs), require a nasogastric (NG) or orogastric (OG) tube inserted through the nose or mouth. These tubes are then pushed down the esophagus and into the stomach.

With an inserted NG/OG apparatus, stomach contents may be suctioned through the tube and/or food and medicines may be administered through the tube and into the stomach. In addition, an esophageal thermometer may be inserted into a patient's esophagus for core temperature monitoring. Traditionally, the NG/OG tube and the standalone thermometer are separate devices which are positioned adjacent to each other in the patient's esophagus.

Once the NG/OG tube is inserted in the patient's stomach, a freestanding adapter is normally used to couple it to dedicated suction tubing. The adapter is small by design, colorless, and is always at risk of falling from the packaging material upon opening. This frequently results in its loss even before initial coupling to the suctioning tube. The same risk exists during suctioning. Oftentimes, the adapter dislodges from the suction apparatus and becomes misplaced at the most inopportune time, and may result in gastric contents leaking in unwanted places. Furthermore, during suctioning, the adapter is constantly at risk for loss. To avoid this, practitioners may tape the adapter to the body of the suction tube or insert it into the vent port of the apparatus. Both of these make-shift solutions are inconvenient and carry the same inherent risk of being lost.

Thus is the reasoning for coupling the adapter to the NG/OG tube proper. This will prevent its loss at a critical juncture. Preventing the adapter's loss will save institutions money by ensuring health care providers do not have to open a separate package just to retrieve a new adapter. It is also desirable to have an NG/OG tube with an internal or integrated temperature sensor so as to eliminate a secondary apparatus placement in the patient's esophagus. Further, it is desirable to have an NG/OG tube temperature sensing apparatus combined to which the adapter is tethered thus eliminating its apparent loss that invariably necessitate the use of a new NG/OG tube because of a missing adapter.

### SUMMARY OF THE INVENTION

An NG/OG tube temperature sensing apparatus according to the present invention for use as mentioned above. The device is as follows: an NG/OG tube having a proximal end opposite a distal end designed in a tubular configuration. The NG/OG tube is flexibly designed and includes multiple apertures evenly spaced at the distal end to facilitate suctioning. An integrated esophageal temperature sensor proximately located thereof and a temperature monitoring element operatively connected to and displaced from the temperature sensor.

The temperature monitoring element is externally located on the NG/OG tube and in communication with the sensor element through wires. The temperature monitoring element may be electric connected to a vital signs monitor or the like. The apparatus includes a tethered adapter for coupling the proximal end of the NG/OG tube via a suction tube and then to the suction canister for collection of stomach contents.

Therefore, a general object of this invention is to provide an NG/OG tube with a tethered adapter that can be connected to a suction apparatus for evacuation of stomach contents, and an integrated temperature sensing apparatus for use in the aforementioned patients and clinical conditions.

Another object of the invention is to provide an NG/OG tube sensing apparatus combination (as described throughout) that limits the amount of probes/tubes inserted through the oro/nasal cavity, thus minimizing the risk of injury or ulceration.

Still another object of this invention is to provide an NG/OG tube temperature sensing apparatus as aforesaid that includes a temperature sensor located approximately ⅔ towards the distal end of the tube in order to measure patient's core temperature.

Yet another object of this invention is to provide NG/OG tube sensing apparatus as aforesaid that includes a temperature sensing element that is selectively connected to an external vital signs monitor.

A further object of this invention is to provide an NG/OG tube with a temperature sensing element as aforesaid in which a tethered adapter is permanently attached to the proximal end of the NG/OG tube so as to eliminate the risk of being dropped and lost at critical junctures of a patient care as well as its financial ramification.

A still further object of this invention is to provide an NG/OG tube sensing apparatus as aforesaid that results in overall cost saving to any health care entity.

Other objects and advantages of the present invention will become apparent from the following description taken in connection with the accompanying drawing, wherein is set forth by way of illustration and example, embodiments of this invention

### BRIEF DESCRIPTION OF THE DRAWINGS

FIG. 1a is a perspective view of a gastric tube temperature sensing apparatus, according to a preferred embodiment of the present invention;

FIG. 1b is an isolated view on an enlarged scale taken from FIG. 1a;

FIG. 1c is an isolated view on an enlarged scale taken from FIG. 1a;

FIG. 1d is an isolated view on an enlarged scale taken from FIG. 1a;

FIG. 2a is a top view of the gastric tube temperature sensing apparatus as in FIG. 1a;

FIG. 2b is a front end view of the apparatus as in FIG. 2a;

FIG. 2c is a sectional view taken along line 2c-2c of FIG. 2b;

FIG. 2d is an isolated view on an enlarged scale taken from FIG. 2c;

FIG. 2e is an isolated view on an enlarged scale taken from FIG. 2c; and

FIG. 2f is an isolated view on an enlarged scale taken from FIG. 2c.

### DESCRIPTION OF THE PREFERRED EMBODIMENT

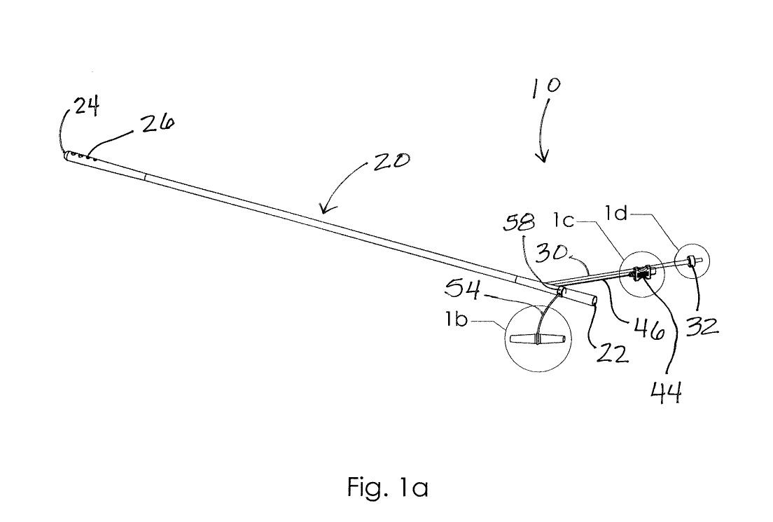

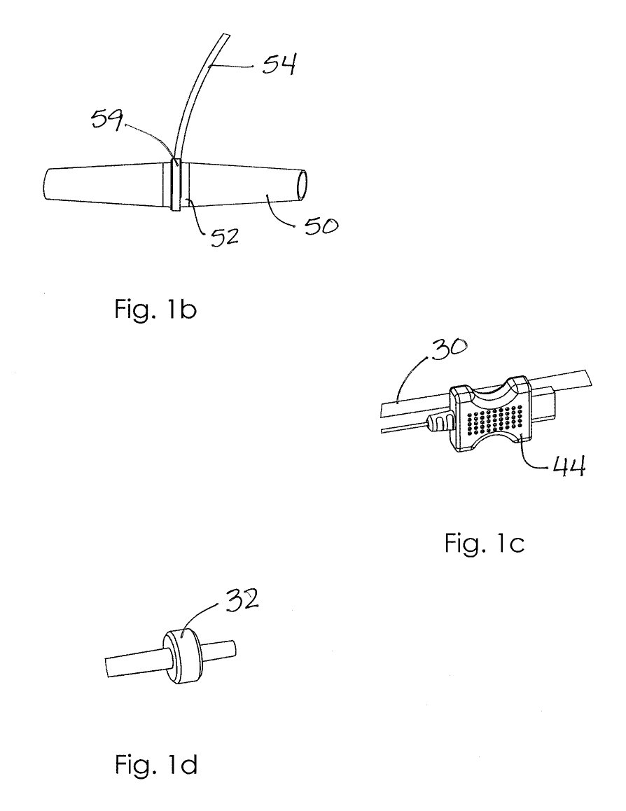

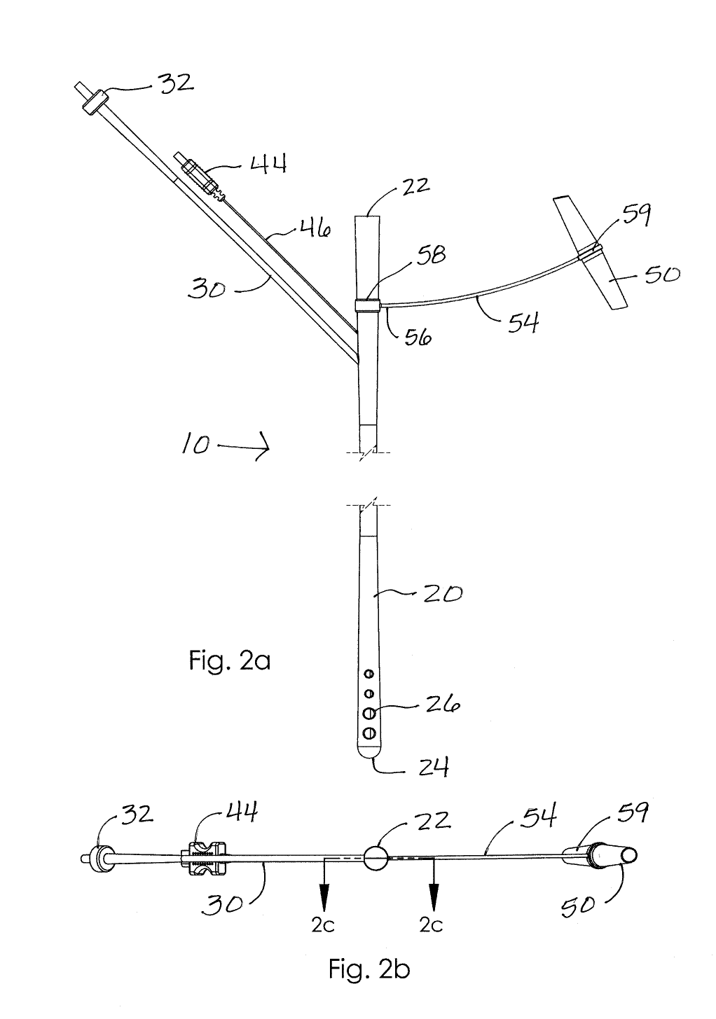

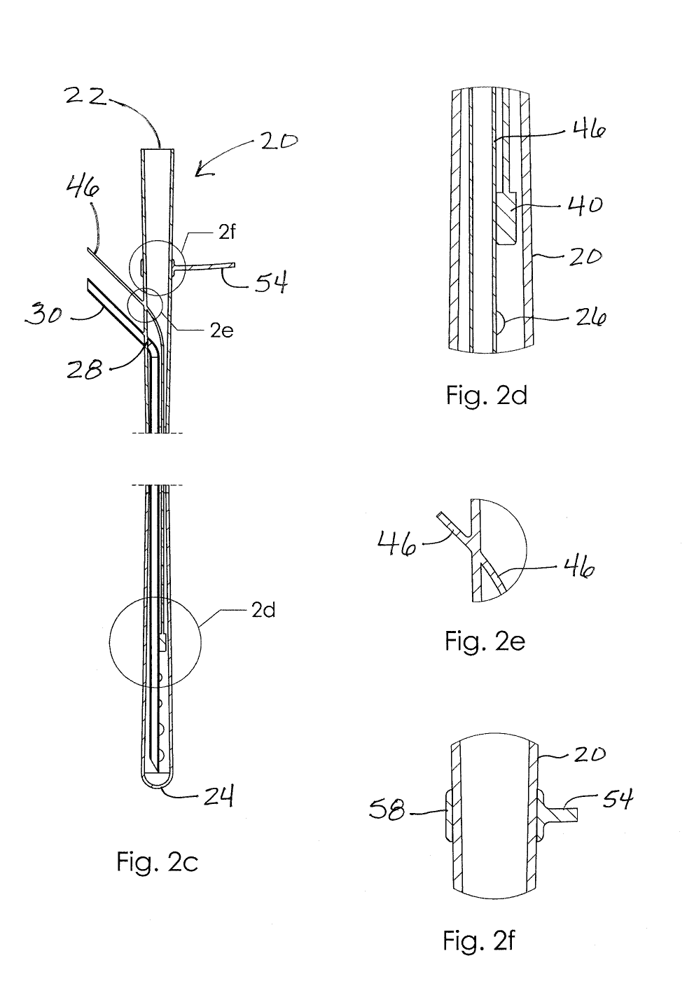

A gastric tube temperature sensing apparatus according to a preferred embodiment of the present invention will now be described in detail along with FIGS. 1 to 2f of the accompanying drawings. The gastric tube temperature sensing apparatus 10 includes a gastric tube 20, an esophageal temperature device 40, and an adapter 50 for connecting the gastric tube 20 to another medical device.

The gastric tube 20 may be a nasogastric tube or an orogastric tube having proximal 22 and distal 24 ends and includes a flexible construction. The gastric tube 20 is constructed so as to be selectively inserted through a patient's nose or mouth until the distal end 24 thereof is situated in the patient's stomach. The gastric tube 20 includes a continuous side wall that defines an interior area. The proximal end 22 is open and gives access to the interior area whereas the distal end 24 is closed. However, the side wall of the gastric tube 20 may define a plurality of spaced apart openings 26 proximate the distal end 24 through which stomach contents may be drawn into the interior area and withdrawn, such as by operation of a suction device (not shown), as will be described later. In an embodiment, the side wall of the gastric tube 20 defines an aperture 28.

The esophageal temperature device 40 includes a temperature sensor 42 situated proximate the distal end 24 of the gastric tube 20. In an embodiment, the temperature sensor 42 is positioned upwardly adjacent to the plurality of openings 26 (FIGS. 2c and 2d) so that, in practice, the temperature sensor 42 may be situated inside a patient's esophagus above the stomach. As a result, accurate data concerning a patient's internal core temperature during a surgical or intestinal procedure may be monitored.

The esophageal temperature device 40 may also include a temperature monitoring element 44 in data communication with the temperature sensor 42. In an embodiment, the temperature monitoring element 44 may be configured electronically to receive digital data indicative of a real time temperature reading at the location of the temperature sensor 42. The temperature sensor 42 and temperature monitoring element 44 may be connected with wires 46. The wires 46 may extend through the aperture 28 of the side wall of the gastric tube 20 such that the temperature monitoring element 44 is situated outside of the gastric tube 20 and extending away therefrom (FIGS. 3a and 2c). It is understood that the temperature monitoring element 44 may be electrically connected to an independent medical monitoring device (not shown), such as a vitals device or other another device monitored by an anesthetist during a surgery or gastric procedure.

The gastric tube 20 includes a vent lumen 30 extending from the side wall thereof and having a tubular configuration being in communication with the aperture 28 (FIGS. 1a and 1d). Accordingly, the vent lumen 30 may also be referred to as a vent tube. The vent lumen 30 is in fluid communication with the interior area defined by the gastric tube 20. An anti-reflux valve 32 may be positioned at a distal end of the vent lumen 30 that prevents inadvertent exposure to stomach contents.

The adapter 50 is a device that enables the proximal end 22 of the gastric tube 20 to be connected to a complementary medical device, such as a suction pump (not shown) for the purpose of withdrawing stomach contents. The adapter 50 includes a body member having a generally tubular configuration and having an outer diameter substantially similar to an inner diameter of the gastric tube. More particularly, the body member includes opposed open ends and a continuous side wall extending between the ends. The side wall defines a groove 52 extending radially thereabout, the groove 52 being positioned at about a midpoint between respective ends.

The adapter 50 is releasably coupled to the gastric tube 20 proximate the proximal end 22 with a tether 54 (FIG. 2a). The tether 54 includes a first end 56 coupled to the gastric tube 20 in a friction fit engagement and a second end 59 coupled to the adapter 50. More particularly, the first end 56 of the tether 54 may include a resilient sleeve 58, such as an elastic band, that may slide over the proximal end 22 and be slidably positioned proximate the proximal end 22. Fasteners such as hook and loop fasteners, snaps, clasps, or the like may also work. The configuration of the first end 56 of the tether 54 is especially important in that the gastric tube 20 may be cut to length depending on the distance from a patient's nose or mouth to his stomach. It is important that when the gastric tube 20 is fully inserted, the distal end 24 is situated just inside the stomach. This may be a different length from patient to patient. The sleeve 58 at the first end 56 of the tether 54 may be slipped over the proximal end 22—once cut—and positioned as desired so that the adapter 50 is always proximate the proximal end 22.

Similarly, the second end 59 may include a resilient cord such as an elastic ring that is received tightly in the groove 52 and difficult to dislodge therefrom. Accordingly, it is intended that the adapter 50 will hang from the gastric tube 20 in close proximity to the proximal end 22 so as to be available for engagement therewith when needed. It is understood that the tether 54 prevents the adapter 50 from being lost or unintentionally displaced from the open proximal end 22.

The opposed ends of the adapter 50 are open such that the adapter 50 is a channel for receiving fluids therethrough. The adapter 50 has its smallest external diameter at respective ends, the diameter incrementally increasing toward the groove 52 midway between the ends. Specifically, the outer diameter proximate each end is smaller than the inner diameter of the gastric tube 20 so as to be selectively insertable therein. Then, as the adapter 50 is inserted further, the increasing diameter tightens the friction fit. When one end is inserted, the opposed end is free and configured to be coupled to a complementary medical device such as a suction pump.

In use, the gastric tube temperature sensing apparatus 10 may be used in a traditional intubation procedure, reduces the quantity of other medical equipment needed for the procedure, and eliminates the problem of lost or dropped adapters 50 needed for coupling the gastric tube 20 to other medical devices used in or after the intubation procedure. Specifically, no esophageal thermometer needs to be inserted alongside the gastric tube 20 in that an esophageal temperature device 40 is situated inside the gastric tube 20 in operative data communication with a temperature monitoring element 44. In addition, the adapter 50 is useful for connecting the gastric tube 20 to an external medical device, such as a suction device, and is tethered to the gastric tube 20 proximate the proximal end 22 so as not to become lost or displaced from the proximal end 22.

It is understood that while certain forms of this invention have been illustrated and described, it is not limited thereto except insofar as such limitations are included in the following claims and allowable functional equivalents thereof.

1. A gastric tube temperature sensing apparatus for use during gastric or surgical procedures on a patient, comprising:
a gastric tube having a proximal end opposite a distal end and defining a tubular configuration, said gastric tube having a flexible construction and defining a plurality of spaced apart openings proximate said distal end; and
an esophageal temperature device having a temperature sensor situated in said gastric tube proximate said distal end and having a temperature monitoring element operatively connected to and displaced from said temperature sensor.

2. The gastric tube temperature sensing apparatus as in claim 1, wherein:
said gastric tube defines an aperture displaced from said proximal end;
wherein said temperature sensor is operatively connected to said temperature monitoring element with wires extending internally through said gastric tube, said wires configured to extend through said aperture.

3. The gastric tube temperature sensing apparatus as in claim 2, further comprising a vent lumen in communication with said aperture in said gastric tube, said vent lumen having an anti-reflux valve coupled to a distal end thereof that prevents exposure to gastric contents.

4. The gastric tube temperature sensing apparatus as in claim 1, wherein said temperature monitoring element is selectively connected to a vitals monitor external and displaced from said gastric tube.

5. The gastric tube temperature sensing apparatus as in claim 1, wherein said proximal end of said gastric tube has an open configuration.

6. The gastric tube temperature sensing apparatus as in claim 1, further comprising an adapter coupled to said gastric tube with a tether proximate said proximal end.

7. The gastric tube temperature sensing apparatus as in claim 6, wherein said tether includes a first end removably coupled to said gastric tube in a friction fit engagement and a second end coupled to said adapter.

8. The gastric tube temperature sensing apparatus as in claim 7, wherein said adapter includes a generally cylindrical configuration having opposed ends and a continuous side wall extending therebetween, said side wall defining a groove extending radially thereabout configured to receive said second end of said tether.

9. The gastric tube temperature sensing apparatus as in claim 8, wherein:
said adapter has a tubular construction in which said opposed ends have open configurations, respectively;
said side wall of said adapter has an increasing external diameter extending from at least one of said opposed ends toward said groove such that said adapter is selectively inserted into said proximal end of said gastric tube in a friction fit engagement tube such that an interior area defined by said adapter is in fluid communication with an interior area defined by said gastric tube;
